(19) United States
(12) Patent Application Publication
(10) Pub. No.: US 2026/0166056 A1
(43) Pub. Date: Jun. 18, 2026

(54) TREPROSTINIL FORMULATIONS AND THEIR USES

(71) Applicant: United Therapeutics Corporation, Silver Spring, MD (US)

(72) Inventors: Hitesh Batra, Herndon, VA (US); Patrick Poisson, Chapel Hill, NC (US); Rob Zwolinski, Silver Spring, MD (US)

(73) Assignee: United Therapeutics Corporation, Silver Spring, MD (US)

(21) Appl. No.: 19/417,891

(22) Filed: Dec. 12, 2025

Related U.S. Application Data
(60) Provisional application No. 63/733,580, filed on Dec. 13, 2024.

(51) Int. Cl. A61K 31/5575; A61K 9/00; A61K 9/08; A61K 31/5578; A61K 47/02; A61K 47/10; A61K 47/18

(52) U.S. Cl. CPC A61K 31/5575; A61K 9/0078; A61K 9/08; A61K 31/5578; A61K 47/02; A61K 47/10; A61K 47/186

(57) ABSTRACT

Provided herein is an inhalation formulation comprising a benzalkonium chloride (BKC) preservative and treprostinil or a pharmaceutically acceptable salt thereof. Also provided herein is an inhalation formulation comprising treprostinil or a pharmaceutically acceptable salt thereof and iloprost or a pharmaceutically acceptable salt thereof. Also provided herein is an inhalation device comprising the inhalation formulation and therapeutic methods of use of the inhalation formulation.

### CROSS-REFERENCE TO RELATED APPLICATIONS

This application claims priority under 35 U.S.C. § 119(e) to U.S. Provisional Application No. 63/733,580, filed Dec. 13, 2024, the content of which is hereby incorporated by reference in its entirety.

### FIELD OF THE INVENTION

The present application relates to methods and kits for therapeutic treatment and, more particularly, to therapeutic methods involving administering treprostinil using a metered dose inhaler and related kits.

### BACKGROUND

All blood is driven through the lungs via the pulmonary circulation in order, among other things, to replenish the oxygen which it dispenses in its passage around the rest of the body via the systemic circulation. The flow through both circulations is in normal circumstances equal, but the resistance offered to it in the pulmonary circulation is generally much less than that of the systemic circulation. When the resistance to pulmonary blood flow increases, the pressure in the circulation is greater for any particular flow. The above described condition is referred to as pulmonary hypertension (PH). Generally, pulmonary hypertension is defined through observations of pressures above the normal range pertaining in the majority of people residing at the same altitude and engaged in similar activities.

Pulmonary hypertension may occur due to various reasons and the different entities of pulmonary hypertension were classified based on clinical and pathological grounds in 5 categories according to the latest WHO convention, see e.g. Simonneau G., et al. J. Am. Coll. Cardiol. 2004; 43(12 Suppl S):5S-12S, which is hereby incorporated by reference for its classification of types of PH. Pulmonary hypertension can be a manifestation of an obvious or explicable increase in resistance, such as obstruction to blood flow by pulmonary emboli, malfunction of the heart's valves or muscle in handling blood after its passage through the lungs, diminution in pulmonary vessel caliber as a reflex response to alveolar hypoxia due to lung diseases or high altitude, or a mismatch of vascular capacity and essential blood flow, such as shunting of blood in congenital abnormalities or surgical removal of lung tissue. In addition, certain infectious diseases, such as HIV and liver diseases with portal hypertension may cause pulmonary hypertension. Autoimmune disorders, such as collagen vascular diseases, also often lead to pulmonary vascular narrowing and contribute to a significant number of pulmonary hypertension patients. The cases of pulmonary hypertension remain where the cause of the increased resistance is as yet inexplicable are defined as idiopathic (primary) pulmonary hypertension (iPAH) and are diagnosed by and after exclusion of the causes of secondary pulmonary hypertension and are in the majority of cases related to a genetic mutation in the bone morphogenetic protein receptor-2 gene. The cases of idiopathic pulmonary arterial hypertension tend to comprise a recognizable entity of about 40% of patients cared for in large specialized pulmonary hypertension centers. Approximately 65% of the most commonly afflicted are female and young adults, though it has occurred in children and patients over 50. Life expectancy from the time of diagnosis is short without specific treatment, about 3 to 5 years, though occasional reports of spontaneous remission and longer survival are to be expected given the nature of the diagnostic process. Generally, however, disease progress is inexorable via syncope and right heart failure and death is quite often sudden.

Pulmonary hypertension refers to a condition associated with an elevation of pulmonary arterial pressure (PAP) over normal levels. In humans, a typical mean PAP is approximately 12-15 mm Hg. Pulmonary hypertension, on the other hand, can be defined as mean PAP above 25 mmHg, assessed by right heart catheter measurement. Pulmonary arterial pressure may reach systemic pressure levels or even exceed these in severe forms of pulmonary hypertension. When the PAP markedly increases due to pulmonary venous congestion, i.e. in left heart failure or valve dysfunction, plasma can escape from the capillaries into the lung interstitium and alveoli. Fluid buildup in the lung (pulmonary edema) can result, with an associated decrease in lung function that can in some cases be fatal. Pulmonary edema, however, is not a feature of even severe pulmonary hypertension due to pulmonary vascular changes in all other entities of this disease.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature, which result in a decreased cross-sectional area of the pulmonary blood vessels. This may be caused by, for example, chronic hypoxia, thromboembolism, collagen vascular diseases, pulmonary hypercirculation due to left-to-right shunt, HIV infection, portal hypertension or a combination of genetic mutation and unknown causes as in idiopathic pulmonary arterial hypertension.

Pulmonary hypertension has been implicated in several life-threatening clinical conditions, such as adult respiratory distress syndrome (“ARDS”) and persistent pulmonary hypertension of the newborn (“PPHN”). Zapol et al., Acute Respiratory Failure, p. 241-273, Marcel Dekker, New York (1985); Peckham, J. Ped. 93:1005 (1978). PPHN, a disorder that primarily affects full-term infants, is characterized by elevated pulmonary vascular resistance, pulmonary arterial hypertension, and right-to-left shunting of blood through the patent ductus arteriosus and foramen ovale of the newborn's heart. Mortality rates range from 12-50%. Fox, Pediatrics 59:205 (1977); Dworetz, Pediatrics 84:1 (1989). Pulmonary hypertension may also ultimately result in a potentially fatal heart condition known as “cor pulmonale,” or pulmonary heart disease. Fishman, “Pulmonary Diseases and Disorders” 2^{nd} Ed., McGraw-Hill, New York (1988).

There exists a need for improved inhalation formulations of treprostinil.

### SUMMARY OF THE INVENTION

An embodiment is an inhalation formulation comprising a preservative and treprostinil or a pharmaceutically acceptable salt thereof and a pharmaceutically acceptable carrier, wherein the treprostinil is at a concentration of about 1 mg/mL to about 25 mg/mL, and wherein the formulation is in a solution form. In some embodiments, the inhalation formulation further comprises an additional active agent for treating a cardiopulmonary disorder, preferably iloprost or a pharmaceutically acceptable salt thereof. In an embodiment, a soft mist inhaler comprising a removable cartridge comprising the formulation is used.

An embodiment is an inhalation formulation comprising treprostinil or a pharmaceutically acceptable salt thereof at a concentration of about 1.2 mg/mL to about 12 mg/mL, benzalkonium chloride at a concentration of about 0.01% (w/w) to about 0.02% (w/w), sodium hydroxide, sodium chloride, sodium citrate dihydrate or sodium citrate, and water. In some embodiments, the inhalation formulation further comprises an additional active agent for treating a cardiopulmonary disorder, preferably iloprost or a pharmaceutically acceptable salt thereof.

An embodiment is a nebulizer or soft mist inhaler comprising the inhalation formulation.

An embodiment is a method of treating a cardiopulmonary disorder comprising administering an effective amount of the inhalation formulation to a patient in need thereof.

### BRIEF DESCRIPTION OF THE DRAWINGS

FIG. 1 shows a 3-D representation of formulations comprising iloprost and treprostinil at different concentrations and 2 different pH levels.

FIG. 2A and FIG. 2B show plots of AUCo-s versus Cₘₐₓ for Treprostinil soft mist inhaler (SMI) (FIG. 2A) and Tyvaso Nebulizer (FIG. 2B).

FIG. 3A and FIG. 3B show comparisons of CL/F between Treprostinil SMI (N=49 FIG. 3A) and Tyvaso Nebulizer (N=98 FIG. 3B).

FIG. 4 shows comparative bioavailability using CL/F for All Participants at All Doses for Tyvaso Nebulizer vs Treprostinil SMI.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined. Additional definitions are set forth throughout the detailed description.

All numerical designations, e.g., pH, temperature, time, concentration, and molecular weight, including ranges, are approximations which are varied (+) or (−) by increments of 0.05%, 1%, 2%, 5%, 10% or 20%. It is to be understood, although not always explicitly stated that all numerical designations are optionally preceded by the term “about.” It also is to be understood, although not always explicitly stated, that the reagents described herein are merely exemplary and that equivalents of such are known in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the present technology, subject to any specifically excluded limit in the stated range. Where the stated range includes one or both of the limits, ranges excluding either or both of those included limits are also included in the present technology.

“Subject” refers to an animal, such as a mammal (including a human), that has been or will be the object of treatment, observation or experiment. “Subject” and “patient” may be used interchangeably, unless otherwise indicated. The methods described herein may be useful in human therapy and/or veterinary applications. In some embodiments, the subject is a mammal. In some embodiments, the subject is a human.

“Pulmonary hypertension” refers to all forms of pulmonary hypertension, WHO Groups 1-5. A preferred embodiment relates to pulmonary arterial hypertension, also referred to as PAH, which encompasses WHO Group 1 pulmonary hypertension. PAH includes idiopathic, heritable, drug- or toxin-induced, and persistent pulmonary hypertension of the newborn (PPHN). “Pulmonary hypertension” includes pulmonary arterial hypertension (“PAH”) (Group 1) in all its forms, including idiopathic and heritable PAH. Pulmonary hypertension includes patients with NYHA Functional Class III symptoms. “Pulmonary hypertension” includes pulmonary hypertension due to left heart disease (Group 2). “Pulmonary hypertension” includes pulmonary hypertension due to lung disease (Group 3). “Pulmonary hypertension” includes thromboembolic pulmonary hypertension (Group 4). “Pulmonary hypertension” includes pulmonary hypertension secondary to other conditions, such as sarcoidosis, sickle cell anemia, chronic hemolytic anemia, splenectomy, and certain metabolic disorders. (Group 5). Generally, the methods of treatment described herein are most applicable to PAH (Group 1) and Group 3 pulmonary hypertension, including PH-ILD.

Treprostinil potassium can provide enhanced control of osmolality as compared with treprostinil and other treprostinil salts in a formulation.

The inhalation formulations may further comprise a preservative. In one aspect the preservative is benzalkonium chloride. In other embodiments, the preservative can be EDTA or chlorobutanol.

The inhalation formulations may also comprise a second prostacyclin. In one aspect the second prostacyclin is iloprost. The combination of treprostinil and iloprost—both prostacyclin analogs—could have a synergistic effect on IP receptors potentially enhancing vasodilation and reducing pulmonary arterial pressure more effectively than either compound alone.

According one embodiment, the present invention relates to an inhalation formulation comprising a preservative and treprostinil or a pharmaceutically acceptable salt thereof and a pharmaceutically acceptable carrier, wherein the treprostinil is at a concentration of about 1 μg/mL to about 25 mg/mL, and wherein the formulation is in a solution form.

According to another embodiment, the present invention relates to an inhalation formulation comprising a benzalkonium chloride (BKC) preservative and treprostinil or a pharmaceutically acceptable salt thereof and a pharmaceutically acceptable carrier, wherein the treprostinil is at a concentration of about 1 mg/mL to about 25 mg/mL, and wherein the formulation is in a solution form. In other embodiments, the preservative can be EDTA or chlorobutanol.

According to another embodiment, the present invention relates to an inhalation formulation comprising treprostinil or a pharmaceutically acceptable salt thereof at a concentration of about 1.2 mg/mL to about 12 mg/mL, BKC at a concentration of about 0.01 (w/w) to about 0.02 (w/w), or any value or range therein between, sodium hydroxide, sodium chloride, sodium citrate dihydrate or sodium citrate, and water.

According to yet another embodiment, the present invention relates to an inhalation formulation comprising treprostinil potassium at a concentration of about 1.2 mg/mL to about 12 mg/mL and BKC at a concentration of about 0.01 (w/w) to about 0.02 (w/w), or any value or range therein between.

According to another embodiment, the present invention relates to an inhalation formulation comprising treprostinil or a pharmaceutically acceptable salt thereof at a concentration of about 1.2 mg/mL to about 12 mg/mL and EDTA or BKC at a concentration of about 0.01 (w/w) to about 0.5 (w/w) or any value or range therein between.

According to a further embodiment, the present invention relates to an inhalation formulation comprising treprostinil potassium at a concentration of about 1.2 mg/mL to about 12 mg/mL and metacresol at a concentration of about 1 mg/mL to about 10 mg/mL.

According to yet a further embodiment, the present invention relates to an inhalation formulation comprising iloprost or a pharmaceutically acceptable salt thereof and treprostinil or a pharmaceutically acceptable salt thereof, wherein the concentration of iloprost ranges from about 10 μg/mL to about 500 μg/mL, and wherein the concentration of treprostinil ranges from about 200 μg/mL to about 4000 μg/mL. In some aspects, the concentration of iloprost ranges from about 200 μg/mL to about 500 μg/mL and the concentration of treprostinil ranges from about 200 μg/mL to about 4000 μg/mL. In some aspects, the concentration of iloprost ranges from about 100 μg/mL to about 200 μg/mL and the concentration of treprostinil ranges from about 200 μg/mL to about 4000 μg/mL. The inhalation formulation comprising treprostinil and iloprost may further comprise the preservative benzalkonium chloride.

The present invention also relates to inhalation devices comprising the inhalation formulations described herein. In one aspect, the inhalation device is a soft mist inhaler.

The present invention also relates to therapeutic methods of use of the inhalation formulations described herein. The inhalation formulation may be used to treat a cardiopulmonary disorder. In one aspect, the cardiopulmonary disorder is selected from pulmonary hypertension and pulmonary fibrosis.

### Treprostinil

Treprostinil is also used for the treatment of pulmonary arterial hypertension. Treprostinil is a synthetic analog of prostacyclin (PGI₂) having the following structure:

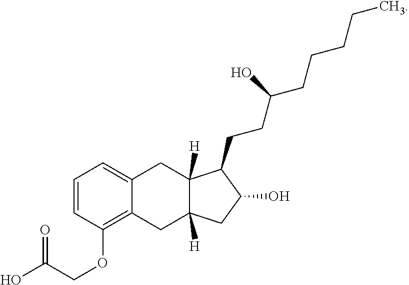 [SMILES: CCCCC[C@H](O)CC[C@@H]1[C@H]2Cc3cccc(OCC(=O)O)c3C[C@H]2C[C@H]1O]

Treprostinil is the active ingredient in Remodulin® (treprostinil) Injection, Tyvaso® (treprostinil) Inhalation Solution, and Orenitram® (treprostinil) Extended-Release Tablets.

Treprostinil was described in U.S. Pat. No. 4,306,075. Methods of making treprostinil and other prostacyclin derivatives are described, for example, in Moriarty, et al., J. Org. Chem. 2004, 69, 1890-1902, Drug of the Future, 2001, 26(4), 364-374, U.S. Pat. Nos. 6,441,245, 6,528,688, 6,700,025, 6,809,223, 6,756,117, 8,461,393, 8,481,782; 8,242,305, 8,497,393, 8,940,930, 9,029,607, 9,156,786 and 9,388,154 9,346,738; U.S. Published Patent Applications Nos. 2012-0197041, 2013-0331593, 2014-0024856, 2015-0299091, 2015-0376106, 2016-0107973, 2015-0315114, 2016-0152548, and 2016-0175319; PCT Application Publications No. WO2016/0055819 and WO2016/081658.

Various uses and/or various forms of treprostinil are disclosed, for example, in U.S. Pat. Nos. 5,153,222, 5,234,953, 6,521,212, 6,756,033, 6,803,386, 7,199,157, 6,054,486, 7,417,070, 7,384,978, 7,879,909, 8,563,614, 8,252,839, 8,536,363, 8,410,169, 8,232,316, 8,609,728, 8,350,079, 8,349,892, 7,999,007, 8,658,694, 8,653,137, 9,029,607, 8,765,813, 9,050,311, 9,199,908, 9,278,901, 8,747,897, 9,358,240, 9,339,507, 9,255,064, 9,278,902, 9,278,903, 9,758,465; 9,422,223; 9,878,972; 9,624,156; 10,716,793; U.S. Published Patent Applications Nos. 2009-0036465, 2008-0200449, 2008-0280986, 2009-0124697, 2014-0275616, 2014-0275262, 2013-0184295, 2014-0323567, 2016-0030371, 2016-0051505, 2016-0030355, 2016-0143868, 2015-0328232, 2015-0148414, 2016-0045470, 2016-0129087, 2017-0095432; 2018-0153847 and PCT Application Publications Nos. WO00/57701, WO20160105538, WO2016038532, WO2018/058124.

The present invention extends to methods of using physiologically acceptable salts of Treprostinil, as well as non-physiologically acceptable salts of Treprostinil that may be used in the preparation of the pharmacologically active compounds of the invention.

“Pharmaceutically acceptable salts” of treprostinil are physiologically acceptable salts, as well as non-physiologically acceptable salts of treprostinil. The term pharmaceutically acceptable salt of Treprostinil refers to a salt of Treprostinil with an inorganic base, organic base, inorganic acid, organic acid, or basic or acidic amino acid. Salts of inorganic bases can be, for example, salts of alkali metals such as sodium or potassium; alkaline earth metals such as calcium and magnesium or aluminum; and ammonia. Salts of organic bases can be, for example, salts trimethylamine, triethylamine, pyridine, picoline, ethanolamine, diethanolamine, and triethanolamine. Salts of inorganic acids can be, for example, salts of hydrochloric acid, hydroboric acid, nitric acid, sulfuric acid, and phosphoric acid. Salts of organic acids can be, for example, salts of formic acid, acetic acid, trifluoroacetic acid, fumaric acid, oxalic acid, lactic acid, tartaric acid, maleic acid, citric acid, succinic acid, malic acid, methanesulfonic acid, benzenesulfonic acid, and p-toluenesulfonic acid. Salts of basic amino acids can be, for example, salts of arginine, lysine and ornithine. Salts of acidic amino acids can include, for example, salts of aspartic acid and glutamic acid. Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

Preferred pharmaceutically acceptable salts are disclosed, for example, in US patent application publication No. 20050085540. Specific examples of treprostinil salts may include treprostinil diethanolamine; treprostinil tromethamine, treprostinil arginine; treprostinil lysine salt, treprostinil N-methylglucamine, treprostinil magnesium, treprostinil ammonium; treprostinil potassium, treprostinil calcium, treprostinil ethylenediamine, treprostinil choline, treprostinil tris(hydroxymethyl)aminomethane (treprostinil TRIS), treprostinil procaine, treprostinil benzathine, treprostinil sodium, as well as the lysine, arginine and potassium salts of treprostinil.

### Iloprost

Iloprost (tradenames: Ventavis®, Ilomedine) is used to treat pulmonary arterial hypertension (PAH), scleroderma, Raynaud's phenomenon and other diseases in which the blood vessels are constricted and blood cannot flow to the tissues. Iloprost was developed by the pharmaceutical company Schering AG and is marketed by Bayer Schering Pharma AG in Europe and Actelion Pharmaceuticals in the USA. Iloprost is a synthetic analogue of prostacyclin PGI₂ having the following structure:

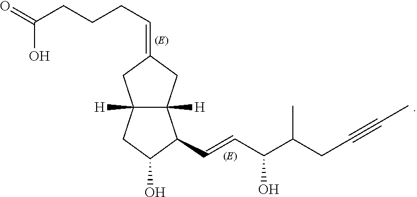 [SMILES: CC#CCC(C)[C@H](O)/C=C/[C@@H]1[C@H]2C/C(=C/CCCC(=O)O)C[C@H]2C[C@H]1O]

In the U.S., iloprost (Ventavis®) solution is approved for inhalation using the I-Neb AAD or Prodose AAD delivery systems. In Europe, iloprost as Ventavis has been approved for use with two compressed air nebulizers with AAD delivery systems (Halolite and Prodose) as well as with two ultrasonic nebulizers, Ventaneb and I-Neb.

“Pharmaceutically acceptable salts” of iloprost are physiologically acceptable salts, as well as non-physiologically acceptable salts of iloprost. Thus iloprost can form a salt with a metal, such as an alkali metal or an alkali earth metal (e.g., Na+, Li+, K+, Ca2+, Mg2+, Zn2+), ammonia or an organic amine (e.g., dicyclohexylamine, trimethylamine, triethylamine, pyridine, picoline, ethanolamine, diethanolamine, triethanolamine, tromethamine) or basic amino acids (e.g., arginine, lysine, histidine and ornithine). Such salts may be prepared in situ during isolation and purification of the compounds or by separately reacting the purified compound in its free acid form with a suitable base, respectively, and isolating the salt thus formed.

### Formulations

In the formulations described herein, the carrier may be a solid or a liquid, or both. The carrier must, of course, be acceptable in the sense of being compatible with any other ingredients in the formulation and must not be deleterious to the subject. In one aspect the carrier is suitable for administration with an inhalation device. The carrier may be included in the formulation to be aerosolized, atomized, or nebulized. In one aspect, the carrier may be sterile water.

In one aspect the carrier in the formulations described herein is a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, or solvent encapsulating material. Pharmaceutically acceptable carriers include saline, aqueous buffer solutions, solvents and/or dispersion media. The use of such carriers are well known in the art.

In the formulations described herein, buffers including hydrochloric acid, sodium hydroxide, sodium citrate dihydrate, sodium citrate, tris, or a combination may be used.

In the formulations described herein, solubilizers including polysorbate 80 or propylene glycol may be used.

In the formulations described herein, an osmotic agent such as sodium chloride may be used. Osmotic agents are well known to those skilled in the art. Osmotic agents may be simple sugars such as sucrose, xylitol, glucose, lactose; salts such as sodium chloride, potassium chloride; low molecular weight hydrophilic polymers such as cellulose ethers, maltodextrins, and cyclodextrins.

The formulations of the invention may be prepared by any of the well-known techniques of pharmacy for admixing the components.

### Inhalation Formulation Comprising a Preservative

According to some embodiments, the present invention relates to an inhalation formulation comprising treprostinil or a pharmaceutically acceptable salt thereof and a preservative, wherein the treprostinil is at a concentration of about 1 to about 25 mg/mL. In some aspects the preservative is benzalkonium chloride (BKC) or metacresol. In other embodiments, the preservative can be EDTA or chlorobutanol.

According to some embodiments, the present invention relates to an inhalation formulation comprising a benzalkonium chloride (BKC) preservative and treprostinil or a pharmaceutically acceptable salt thereof, wherein the treprostinil is at a concentration of about 1 to about 25 mg/mL, and wherein the formulation is in a solution form. In one aspect the inhalation formulation further comprises a pharmaceutically acceptable carrier.

In some aspects, the treprostinil or pharmaceutically acceptable salt thereof is at a concentration of about 1.2 to about 12 mg/mL. In some aspects, the treprostinil is at a concentration of about 1.0 mg/mL, or about 1.1 mg/mL, or about 1.2 mg/mL, or about 1.3 mg/mL, or about 1.4 mg/mL, or about 1.5 mg/mL, or about 1.6 mg/mL, or about 1.7 mg/mL, or about 1.8 mg/mL, or about 1.9 mg/mL, or about 2.0 mg/mL, or about 2.5 mg/mL, or about 3.0 mg/mL, or about 3.5 mg/mL, or about 4.0 mg/mL, or about 4.5 mg/mL, or about 5.0 mg/mL, or about 5.5 mg/mL, or about 6.0 mg/mL, or about 6.5 mg/mL, or about 7.0 mg/mL, or about 7.5 mg/mL, or about 8.0 mg/mL, or about 8.5 mg/mL, or about 9.0 mg/mL, or about 9.5 mg/mL, or about 10.0 mg/mL, or about 10.5 mg/mL, or about 11.0 mg/mL, or about 11.5 mg/mL, or about 12.0 mg/mL.

In some aspects the pharmaceutically acceptable treprostinil salt is treprostinil sodium or treprostinil potassium. In some aspects the pharmaceutically acceptable treprostinil salt is a different treprostinil salt.

The BKC preservative may be at a concentration of about 0.005% (w/w) to about 0.20% (w/w). In some aspects the BKC preservative is at a concentration of about 0.01% (w/w) to about 0.02% (w/w), or any range or value therein between. In some aspects, the BKC preservative is at a concentration of about 0.005% (w/w), about 0.01% (w/w), about 0.015% (w/w), about 0.02% (w/w), about 0.025% (w/w), about 0.03% (w/w), about 0.035% (w/w), about 0.04% (w/w), about 0.045% (w/w), about 0.05% (w/w), about 0.055% (w/w), about 0.06% (w/w), about 0.065% (w/w), about 0.07% (w/w), about 0.075% (w/w), about 0.08% (w/w), about 0.085% (w/w), about 0.09% (w/w), about 0.095% (w/w), about 0.1% (w/w), about 0.11% (w/w), about 0.12% (w/w), about 0.13% (w/w), about 0.14% (w/w), about 0.15% (w/w), about 0.16% (w/w), about 0.17% (w/w), about 0.18% (w/w), about 0.19% (w/w), or about 0.20% (w/w), or any range or value therein between. In one aspect the BKC preservative is at a concentration of about 0.01% (w/w). In one aspect the BKC preservative is at a concentration of 0.02% (w/w). In other embodiments, the preservative can be EDTA or chlorobutanol. The chlorobutanol can be at a concentration of about 0.005% (w/w), about 0.01% (w/w), about 0.015% (w/w), about 0.02% (w/w), about 0.025% (w/w), about 0.03% (w/w), about 0.035% (w/w), about 0.04% (w/w), about 0.045% (w/w), about 0.05% (w/w), about 0.055% (w/w), about 0.06% (w/w), about 0.065% (w/w), about 0.07% (w/w), about 0.075% (w/w), about 0.08% (w/w), about 0.085% (w/w), about 0.09% (w/w), about 0.095% (w/w), about 0.1% (w/w), about 0.11% (w/w), about 0.12% (w/w), about 0.13% (w/w), about 0.14% (w/w), about 0.15% (w/w), about 0.16% (w/w), about 0.17% (w/w), about 0.18% (w/w), about 0.19% (w/w), or about 0.20% (w/w), 0.30% (w/w), 0.4% (w/w), 0.5% (w/w), or any range or value therein between. In one aspect the EDTA is at a concentration of less than 0.5% (w/w). The EDTA can be at a concentration of about 0.005% (w/w), about 0.01% (w/w), about 0.015% (w/w), about 0.02% (w/w), about 0.025% (w/w), about 0.03% (w/w), about 0.035% (w/w), about 0.04% (w/w), about 0.045% (w/w), about 0.05% (w/w), or any value or range therein between. In one aspect the EDTA is at a concentration of less than 0.05% (w/w). In one aspect, BKC, EDTA, and chlorobutanol are used in combination at any one of the concentrations or ranges provided herein.

In some aspects the metacresol preservative is at a concentration of about 3 mg/mL. In some aspects the metacresol preservative is at a concentration of about 1 mg/mL to about 10 mg/mL. In some aspects, the metacresol preservative is at a concentration of about 1 mg/mL, or about 2 mg/mL, or about 3 mg/mL, or about 4 mg/mL, or about 5 mg/mL, or about 6 mg/mL, or about 7 mg/mL, or about 8 mg/mL, or about 9 mg/mL, or about 10 mg/mL.

According to one embodiment, the present invention relates to an inhalation formulation comprising treprostinil or a pharmaceutically acceptable salt thereof at a concentration of about 1.2 mg/mL to about 12 mg/mL, BKC at a concentration of about 0.01 (w/w) to about 0.02 (w/w), sodium hydroxide, sodium chloride, and sodium citrate dihydrate or sodium citrate. In one aspect, the inhalation formulation further comprises a carrier. In one aspect, the carrier a pharmaceutically acceptable carrier.

According to one embodiment, the present invention relates to an inhalation formulation comprising treprostinil potassium at a concentration of about 1.2 mg/mL to about 12 mg/mL and BKC at a concentration of about 0.01 (w/w) to about 0.02 (w/w). In one aspect, the inhalation formulation further comprises a carrier. In one aspect, the carrier is a pharmaceutically acceptable carrier.

According to yet another embodiment, the present invention relates to an inhalation formulation comprising treprostinil potassium at a concentration of about 1.2 mg/mL to about 12 mg/mL and metacresol at a concentration of about 1 mg/mL to about 10 mg/mL. In one aspect, the inhalation formulation further comprises a carrier. In one aspect, the carrier is a pharmaceutically acceptable carrier.

In some aspects, the inhalation formulation further comprises a second prostacyclin. In one aspect, the second prostacyclin is iloprost or a pharmaceutically acceptable salt thereof. According to some aspects, the concentration of iloprost in the inhalation formulation ranges from about 100 μg/mL to about 500 g/mL.

In some aspects the pH of the inhalation formulation is about 5.5 to about 9.0. In one aspect, the pH of the inhalation formulation is about 6.6. In one aspect, the pH of the inhalation formulation is about 6.3. In one aspect, the pH of the solution is about 8.4. In another aspect, the pH of the inhalation formulation is about 7.5.

In some aspects the viscosity of the inhalation formulation is about 0.8 cP to about 1.2 cP.

The inhalation formulation can be administered using any suitable means including, but not limited to, a nebulizer, a metered-dose inhaler, some other form of inhaler, or as a soft mist.

### Treprostinil-Iloprost Combination Inhalation Formulation

According to one embodiment, the present invention relates to a treprostinil-iloprost combination inhalation formulation comprising iloprost or a pharmaceutically acceptable salt thereof and treprostinil or a pharmaceutically acceptable salt thereof, wherein the concentration of iloprost ranges from about 10 μg/mL to about 500 μg/mL, and wherein the concentration of treprostinil ranges from about 200 μg/mL to about 4000 μg/mL. In one aspect, the concentration of iloprost ranges from about 10 μg/mL to about 50 μg/mL and the concentration of treprostinil ranges from about 200 μg/mL to about 4000 μg/mL. In yet another aspect, the concentration of iloprost ranges from about 100 μg/mL to about 200 μg/mL and the concentration of treprostinil ranges from about 200 μg/mL to about 4000 μg/mL. In still another aspect, the concentration of iloprost ranges from about 200 μg/mL to about 500 μg/mL and the concentration of treprostinil ranges from about 200 μg/mL to about 4000 μg/mL. In some aspects, the concentration of iloprost ranges from about 100 μg/mL to about 500 μg/mL, or is selected from 30 μg/mL, 140 μg/mL, and 300 μg/mL.

In some aspects, the treprostinil-iloprost combination inhalation formulation further comprises benzalkonium chloride (BKC). In one aspect, the BKC is at a concentration of about 0.01% (w/w) to about 0.02% (w/w). In some aspects, the BKC preservative is at a concentration of about 0.005% (w/w), about 0.01% (w/w), about 0.015% (w/w), about 0.02% (w/w), about 0.025% (w/w), about 0.03% (w/w), about 0.035% (w/w), about 0.04% (w/w), about 0.045% (w/w), about 0.05% (w/w), about 0.055% (w/w), about 0.06% (w/w), about 0.065% (w/w), about 0.07% (w/w), about 0.075% (w/w), about 0.08% (w/w), about 0.085% (w/w), about 0.09% (w/w), about 0.095% (w/w), about 0.1% (w/w), about 0.11% (w/w), about 0.12% (w/w), about 0.13% (w/w), about 0.14% (w/w), about 0.15% (w/w), about 0.16% (w/w), about 0.17% (w/w), about 0.18% (w/w), about 0.19% (w/w), or about 0.20% (w/w). In one aspect, the BKC preservative is at a concentration of about 0.01% (w/w). In one aspect, the BKC preservative is at a concentration of about 0.02% (w/w). In other embodiments, the preservative can be EDTA or chlorobutanol present at the same concentrations.

In some aspects the treprostinil-iloprost combination inhalation formulation further comprises metacresol. In one aspect, the metacresol is at a concentration of about 1 mg/mL to about 10 mg/mL.

In some aspects the pH of the treprostinil-iloprost inhalation formulations described herein is about 5.5 to about 9.0. In one aspect, the pH of the inhalation formulation is about 6.6. In one aspect, the pH of the inhalation formulation is about 6.3. In one aspect, the pH of the solution is about 8.4. In another aspect, the pH of the inhalation formulation is about 7.5.

In some aspects the viscosity of the treprostinil-iloprost inhalation inhalation formulations described herein is about 0.8 cP to about 1.2 cP.

### Methods of Use

Treprostinil and/or other prostacyclins can be administered by inhalation, which in the present context refers to the delivery of the active ingredient or a combination of active ingredients through a respiratory passage, wherein the subject in need of the active ingredient(s) through the subject's airways, such as the subject's nose or mouth.

A metered dose inhaler in the present context means a device capable of delivering a metered or bolus dose of respiratory drug, such as treprostinil, to the lungs. One example of the inhalation device can be a pressurized metered dose inhaler, a device which produces the aerosol clouds for inhalation from solutions and/or suspensions of respiratory drugs in chlorofluorocarbon (CFC) and/or hydrofluoroalkane (HFA) solutions.

The metered dose inhaler can be a soft mist inhaler (SMI), in which the aerosol cloud containing a respiratory drug can be generated by passing a solution containing the respiratory drug through a nozzle or series of nozzles. The aerosol generation can be achieved in SMI, for example, by mechanical, electromechanical or thermomechanical process. Examples of soft mist inhalers include the Respimat® Inhaler (Boeringer Ingelheim GmbH), the AERx® Inhaler (Aradigm Corp.), the Mystic™ Inhaler (Ventaira Pharmaceuticals, Inc) and the Aira™ Inhaler (Chrysalis Technologies Incorporated). For a review of soft mist inhaler technology, see e.g. M. Hindle, The Drug Delivery Companies Report, Autumn/Winter 2004, pp. 31-34. The aerosol for SMI can be generated from a solution of the respiratory drug further containing pharmaceutically acceptable excipients. In the present case, the respiratory drug(s) can be formulated in SMI as a solution. The solution can be, for example, a solution of treprostinil in water, ethanol or a mixture thereof. Preferably, the diameter of the treprostinil-containing aerosol particles is less than about 10 microns, or less than about 5 microns, or less than about 4 microns.

According to one embodiment, the present invention relates to a soft mist inhaler comprising an effective amount of any of the inhalation formulations described herein.

According to another embodiment is a method of treating a cardiopulmonary disease or disorder comprising administering by inhalation to a patient in need thereof a therapeutically effective single event dose of any of the inhalation formulations described herein. In one aspect the administering is performed with a soft mist inhalation device.

In some aspects, the cardiopulmonary disease is selected from pulmonary hypertension and pulmonary fibrosis. In one aspect the disease is pulmonary hypertension associated with interstitial lung disease. In one aspect the cardiopulmonary disease is idiopathic pulmonary fibrosis (IPF).

A dose or amount of treprostinil or its pharmaceutically acceptable salt by inhalation in a single administering event may vary. In some embodiments, the single administering event dose of treprostinil may be at least 5 μg or at least 6 μg. In some embodiments, the single administering event dose of treprostinil may be from 5 μg to 120 μg or from 18 μg to 120 μg or from 6 μg to 96 μg or from 7.5 μg to 100 μg or 10 μg to 100 μg or 15 μg to 100 μg from 15 μg to 90 μg or from 15 μg to 75 μg or from 30 μg to 75 μg or any value or subrange within these ranges.

In an embodiment, a preferred single administering event of treprostinil comprises inhalation of at least 12 μg or at least 15 μg or at least 18 μg, which is preferably inhaled in one or two breaths. In an embodiment, a patient may titrate up to the highest tolerated amount of treprostinil for a single administering event. In another embodiment, an upper limit for a single administering event of treprostinil comprises up to 18 μg, up to 36 μg, up to 54 μg, up to 72 μg, up to 90 μg, up to 108 μg, up to 126 μg, or up to 144 μg. A patient may perform one administering event once per day, twice per day, 3 times per day, 4 times per day, or more.

A dose or amount of iloprost or its pharmaceutically acceptable salt administered by inhalation in a single administering event may vary. In some embodiments, the single administering event dose of iloprost may be at least 2 μg or at least 2.5 μg. In some embodiments, the single administering event dose of iloprost may be from 2 μg to 10 μg or from 2 μg to 9 μg or from 2.5 μg to 5 μg or any value or subrange within these ranges. In some embodiments, a daily dose of iloprost administered over all administering events of the single day may be less than 15 μg or no more or less than 14 μg or no more or less than 13 μg or no more or less than 12 μg or no more or less than 11 μg or no more or less than 10 μg or no more or less than 9 μg or no more or less than 8 μg or no more or less than 7 μg or no more or less than 6 μg or no more or less than 5 μg.

In some embodiments, administering the pharmaceutical formulation, such as a liquid inhalation formulation, may be performed in a single administering event comprising one breath or more than one breath. In some embodiments, a number of breaths in the single administering event or in the single dose event may not exceed 20 breaths (or inhalations) or 19 breaths (or inhalations) or 18 breaths (or inhalations) or 17 breaths (or inhalations) or 16 breaths (or inhalations) or 15 breaths (or inhalations) or 14 breaths (or inhalations) or 13 breaths (or inhalations) or 12 breaths (or inhalations) or 11 breaths (or inhalations) or 10 breaths (or inhalations) or 9 breaths (or breaths (or inhalations) inhalations) or 8 breaths (or inhalations) or 7 breaths (or inhalations) or 6 breaths (or inhalations) or 5 breaths (or inhalations) or 4 breaths (or inhalations) or 3 breaths (or inhalations) or 2 breaths (or inhalations) or 1 breath (or inhalation).

The total time of a single administering event can be less than 5 minutes, or less than 1 minute, or less than 30 seconds.

Treprostinil can be administered as single administering event one time per day or several times per day, including 2 times per day, 3 times per day, or 4 times per day.

In some embodiments, the method of treatment of cardiopulmonary disease or disorder can further comprise administering at least one supplementary agent.

### Kit

The present invention also provides a kit that includes an inhaler containing an inhalation formulation described herein. Such a kit can further include instructions on how to use the inhaler. Such instructions can include, for example, information on how to coordinate patient's breathing, and actuation of the inhaler. The kit can be used by a subject, such as human being, affected with a disease or condition that can be treated by treprostinil and/or iloprost, such as asthma, pulmonary hypertension, peripheral vascular disease or pulmonary fibrosis.

In some cases, the kit is a kit for treating pulmonary hypertension, that includes (i) a metered dose inhaler containing an inhalation formulation described herein; and (ii) instructions for use of the metered dose inhaler in treating pulmonary hypertension.

As used herein, the phrase “instructions for use” shall mean any FDA-mandated labeling, instructions, or package inserts that relate to the administration of the active ingredient, for treatment of pulmonary hypertension by inhalation. For example, instructions for use may include, but are not limited to, indications for pulmonary hypertension, identification of specific symptoms associated with pulmonary hypertension, that can be ameliorated by Treprostinil, recommended dosage amounts for subjects suffering from pulmonary hypertension and instructions on coordination of individual's breathing and actuation of the metered dose inhaler.

The present invention can be illustrated in more detail by the following example, however, it should be understood that the present invention is not limited to the example.

### EXAMPLES

The following examples are intended to provide those of ordinary skill in the art with a complete disclosure and description of how to make and use the methods and compositions described herein, and are not intended to be limiting.

### Example 1

Applicant developed inhalation formulations of treprostinil. Applicant modified the treprostinil concentration (mg/mL), pH range, preservative, buffers, and solubilizers in the formulations. Applicant also tested the compatibility of filters with the inhalation formulations. The data in Tables 1-6 are a summary of the formulations with different characteristics.

The formulations disclosed herein may be used with inhalation devices, such as soft mist inhalers, to treat cardio-pulmonary diseases including pulmonary arterial hypertension (PAH), pulmonary hypertension-interstitial lung disease (PH-ILD), and any form of pulmonary fibrosis, including idiopathic pulmonary fibrosis (IPF) and progressive pulmonary fibrosis (PPF).

### Methods

Inhalation formulation solutions were prepared by combining the components listed in each table. While each formulation varies, the methods for preparing the formulations follows the same steps, adjusting where necessary for the correct parameter. The method can be sized up accordingly for a larger batch of inhalation formulation solution.

The following is the formulation procedure for a 200 mL batch:
1. Add approximately 100 ml of water into a clean bottle with a magnetic stir bar
2. Add treprostinil. Mix the solution.
3. Add sodium hydroxide. Mix the solution.
4. Add sodium chloride. Mix the solution.
5. Add sodium citrate. Mix the solution.
6. Measure benzalkonium chloride (BKC) in a beaker. Add approximately 5 mL of water to the beaker and add BKC solution to the bottle. Mix the solution.
7. Repeat step 5 two additional times, for a total of three BKC transfer steps.
8. Add water until the solution is approximately 180 grams.
9. Adjust the pH to approximately 6.5-6.7. Mix the solution.
10. Filter the solution using a 0.22 μM PES vacuum filter. Collect the filtered solution in a clean bottle.

TABLE 1
Formulations with different buffers and quantities of BKC (benzalkonium chloride).
Assay values in the bottom row indicate the potency of the formulation, representing
the total content of treprostinil as measured against a reference standard.

| | Formulation: | | | | | | | |
|---|---|---|---|---|---|---|---|---|
| Components | 1 | 2 | 3 | 4 | 5 | 6 | 7 | 8 |
| Treprostinil Conc | 12.86 | 14.49 | 14.50 | 14.43 | 15.00 | 12.10 | 14.06 | 14.04 |
| (mg/mL): | | | | | | | | |
| Treprostinil | 0.03293 | 0.03710 | 0.03714 | 0.03694 | 0.03841 | 0.03098 | 0.03601 | 0.03596 |
| (mmol/mL) | | | | | | | | |
| Sodium hydroxide | 1.05 | 1.17 | 1.17 | 1.13 | 1.17 | 0.96 | 1.13 | 1.18 |
| (mg/mL): | | | | | | | | |
| Sodium hydroxide | 0.02619 | 0.02914 | 0.02916 | 0.02826 | 0.02920 | 0.02396 | 0.02826 | 0.02955 |
| (mmol/mL): | | | | | | | | |
| Eq. of Sodium | 0.795 | 0.785 | 0.785 | 0.765 | 0.760 | 0.774 | 0.785 | 0.822 |
| Hydroxide | | | | | | | | |
| TRIS base | 15.21 | 17.00 | 16.97 | 16.71 | 6.19 | 5.53 | 6.02 | 17.04 |
| (mg/mL): | | | | | | | | |
| Citric Acid | — | — | — | — | 3.31 | 2.68 | 2.86 | 6.44 |
| (mg/mL): | | | | | | | | |
| 1N HCl to pH | 5.97 | 6.58 | 6.96 | 8.00 | 5.98 | 6.61 | 6.99 | 8.00 |
| (final): | | | | | | | | |
| BKC (%): | 0.00 | 0.02 | 0.00 | 0.00 | 0.00 | 0.02 | 0.00 | 0.00 |
| WFI (g): | 139.75 | 125.07 | 125.31 | 127.18 | 123.01 | 149.27 | 126.31 | 124.68 |
| pH: | 5.97 | 6.58 | 6.96 | 8.00 | 5.98 | 6.61 | 6.99 | 8.00 |
| Osmolality | 248.00 | 268.50 | 266.00 | 231.50 | 82.00 | 73.50 | 79.50 | 164.50 |
| (average): | | | | | | | | |
| Viscosity (cP): | 1.10 | 1.10 | 1.04 | 1.10 | 0.98 | 1.16 | 1.04 | 1.23 |
| Assay | 87.07 | 97.55 | 96.60 | 96.18 | 98.52 | 81.20 | 96.34 | 95.69 |

TABLE 2
Formulations with different ionic concentrations. Assay
values in the bottom row indicate the potency of the
formulation, representing the total content of treprostinil
as measured against a reference standard.

| | Formulation: | | | |
|---|---|---|---|---|
| Components | 1 | 2 | 3 | 4 |
| Treprostinil Conc | 9.08 | 9.64 | 9.30 | 9.27 |
| (mg/mL): | | | | |
| Treprostinil | 0.02326 | 0.02469 | 0.02381 | 0.02373 |
| (mmol/mL) | | | | |
| Sodium hydroxide | 1.12 | 1.28 | 1.22 | 1.22 |
| (mg/mL): | | | | |
| Sodium hydroxide | 0.02805 | 0.03189 | 0.03046 | 0.03040 |
| (mmol/mL): | | | | |
| Eq. of Sodium | 1.206 | 1.292 | 1.279 | 1.281 |
| Hydroxide | | | | |
| Sodium chloride | 3.30 | 3.50 | 1.69 | 1.67 |
| (mg/mL): | | | | |
| Sodium citrate | 3.19 | 3.36 | 1.64 | 1.61 |
| (mg/mL): | | | | |
| 1N HCl to pH (final): | 6.62 | 6.54 | 6.64 | 6.63 |
| BKC (%): | 0 | 0.02 | 0.00 | 0.02 |
| WFI (g): | 124.87 | 117.70 | 121.49 | 122.38 |
| pH after adjust: | 6.6 | 6.5 | 6.6 | 6.6 |
| Osmolality | 183 | 188 | 113 | 117 |
| (average): | | | | |
| Viscosity: | 1.10 | 1.23 | 1.16 | 1.04 |
| Assay | 99.21 | 105.01 | 99.67 | 102.93 |

TABLE 3
Formulations with different solubilizers. Assay values in the bottom row indicate the potency of the
formulation, representing the total content of treprostinil as measured against a reference standard.

| | Formulation: | | | | | | | |
|---|---|---|---|---|---|---|---|---|
| Components | 1 | 2 | 3 | 4 | 5 | 6 | 7 | 8 |
| Treprostinil | 24.26 | 24.45 | 16.47 | 16.35 | 24.92 | 24.26 | 15.52 | 6.44 |
| Conc | | | | | | | | |
| (mg/mL): | | | | | | | | |
| Treprostinil | 0.06212 | 0.06260 | 0.04218 | 0.04187 | 0.06381 | 0.06213 | 0.03973 | 0.01649 |
| (mmol/mL) | | | | | | | | |
| Sodium | 1.14 | 1.22 | 1.15 | 1.17 | 1.23 | 1.24 | 1.25 | 1.09 |
| hydroxide | | | | | | | | |
| (mg/mL): | | | | | | | | |
| Sodium | 0.02858 | 0.03045 | 0.02881 | 0.02915 | 0.03081 | 0.03091 | 0.03127 | 0.02734 |
| hydroxide | | | | | | | | |
| (mmol/mL): | | | | | | | | |
| Eq. of Sodium | 0.460 | 0.486 | 0.683 | 0.696 | 0.483 | 0.498 | 0.787 | 1.659 |
| Hydroxide | | | | | | | | |
| Sodium | — | — | 6.52 | 6.52 | — | — | 6.53 | 6.55 |
| chloride | | | | | | | | |
| (mg/mL): | | | | | | | | |
| Sodium | — | — | 6.32 | 6.32 | — | — | 6.33 | 6.35 |
| citrate | | | | | | | | |
| (mg/mL): | | | | | | | | |
| TRIS base | 16.99 | 17.00 | — | — | 17.01 | 17.00 | — | — |
| (mg/mL): | | | | | | | | |
| 1N HCl to pH | 6.6 | 7.0 | 6.6 | 7.1 | 6.6 | 7.0 | 6.6 | 7.0 |
| (final): | | | | | | | | |
| Polysorbate | 0.28 | 0.26 | 0.27 | 0.30 | — | — | — | — |
| 80 (mg/mL): | | | | | | | | |
| Propylene | — | — | — | — | 1.60 | 1.59 | 1.64 | 1.51 |
| glycol | | | | | | | | |
| (mg/mL): | | | | | | | | |
| WFI (g): | 125.12 | 125.12 | 125.00 | 124.92 | 125.00 | 125.08 | 124.76 | 124.50 |
| pH after | 6.6 | 7.0 | 6.6 | 7.1 | 6.6 | 7.0 | 6.6 | 7.0 |
| adjust: | | | | | | | | |
| Osmolality | 230 | 235 | 301 | 303 | 258 | 258 | 321 | 343 |
| (average): | | | | | | | | |
| Viscosity (cP): | 1.35 | 1.23 | 0.98 | 1.16 | 1.16 | 1.04 | 1.04 | 1.10 |
| Assay: | 95.95 | 95.12 | 97.12 | 97.10 | 95.44 | 96.43 | 97.34 | 99.40 |

Table 4A-4B. Formulations filtered with different sterilizing filters. Treprostinil recovery (4A) and BKC recovery (4B). Appropriate filters are necessary to prepare a sterile formulation for inhalation. PES (polyethersulfone), PVDF (polyvinylidene fluoride), and PTFE (polytetrafluoroethylene) filters were tested. BZK is benzalkonium chloride.

A.

| | | | | | | Treprostinil |
|---|---|---|---|---|---|---|
| Theo. Treprostinil | Theo. BKC | | | Treprostinil | Treprostinil | % Diff from |
| Conc. (mg/mL) | Conc. (μg/mL) | Sample ID | Filter Type | (mg/mL) | Recovery (%) | control |
| 1.2168 | 199.7565 | 300431- | | 1.20609 | 99.1 | |
| | | Control-F5 | | | | |
| | | 300431-PES- | PES | 1.21455 | 99.8 | 0.7 |
| | | F5-1 | | | | |
| | | 300431-PES- | | 1.21950 | 100.2 | 1.1 |
| | | F5-2 | | | | |
| | | 300431-PES- | | 1.21879 | 100.2 | 1.1 |
| | | F5-3 | | | | |
| | | 300431- | PVDF | 1.21975 | 100.2 | 1.1 |
| | | PVDF-F5-1 | | | | |
| | | 300431- | | 1.20801 | 99.3 | 0.2 |
| | | PVDF-F5-2 | | | | |
| | | 300431- | | 1.21368 | 99.7 | 0.6 |
| | | PVDF-F5-3 | | | | |
| | | 300431- | PTFE | 1.21688 | 100.0 | 0.9 |
| | | PTFE-F5-1 | | | | |
| | | 300431- | | 1.21447 | 99.8 | 0.7 |
| | | PTFE-F5-2 | | | | |
| | | 300431- | | 1.20309 | 98.9 | 0.2 |
| | | PTFE-F5-3 | | | | |
| 5.4172 | 203.1041 | 300431- | | 5.43703 | 100.4 | |
| | | Control-F6 | | | | |
| | | 300431-PES- | PES | 5.43352 | 100.3 | 0.1 |
| | | F6-1 | | | | |
| | | 300431-PES- | | 5.48581 | 101.3 | 0.9 |
| | | F6-2 | | | | |
| | | 300431-PES- | | 5.48197 | 101.2 | 0.8 |
| | | F6-3 | | | | |
| | | 300431- | PVDF | 5.45893 | 100.8 | 0.4 |
| | | PVDF-F6-1 | | | | |
| | | 300431- | | 5.45502 | 100.7 | 0.3 |
| | | PVDF-F6-2 | | | | |
| | | 300431- | | 5.43704 | 100.4 | 0.0 |
| | | PVDF-F6-3 | | | | |
| | | 300431- | PTFE | 5.45501 | 100.7 | 0.3 |
| | | PTFE-F6-1 | | | | |
| | | 300431- | | 5.43762 | 100.4 | 0.0 |
| | | PTFE-F6-2 | | | | |
| | | 300431- | | 5.41298 | 99.9 | 0.4 |
| | | PTFE-F6-3 | | | | |
| 9.0341 | 198.0798 | 300431- | | 9.01518 | 99.8 | |
| | | Control-F7 | | | | |
| | | 300431-PES- | PES | 9.03129 | 100.0 | 0.2 |
| | | F7-1 | | | | |
| | | 300431-PES- | | 9.09911 | 100.7 | 0.9 |
| | | F7-2 | | | | |
| | | 300431-PES- | | 9.02875 | 99.9 | 0.2 |
| | | F7-3 | | | | |
| | | 300431- | PVDF | 9.08985 | 100.6 | 0.8 |
| | | PVDF-F7-1 | | | | |
| | | 300431- | | 9.15456 | 101.3 | 1.5 |
| | | PVDF-F7-2 | | | | |
| | | 300431- | | 9.07189 | 100.4 | 0.6 |
| | | PVDF-F7-3 | | | | |
| | | 300431- | PTFE | 9.07256 | 100.4 | 0.6 |
| | | PTFE-F7-1 | | | | |
| | | 300431- | | 9.03247 | 100.0 | 0.2 |
| | | PTFE-F7-2 | | | | |
| | | 300431- | | 9.04225 | 100.1 | 0.3 |
| | | PTFE-F7-3 | | | | |
| 9.0341 | 198.0798 | | PES, re-filtered | | | |
| | | | from stock | | | |

B.

| Theo. | Theo. | | | | | | | | | BKC |
|---|---|---|---|---|---|---|---|---|---|---|
| Treprostinil | BKC | | | BKC, | BKC, | BKC, | BKC, | BKC, | BKC | % Diff |
| Conc. | Conc. | Sample | Filter | C10 | C12 | C14 | C16 | Total | Recovery | from |
| (mg/mL) | (μg/mL) | ID | Type | (μg/mL) | (μg/mL) | (μg/mL) | (μg/mL) | (μg/mL) | (%) | control |
| 1.2168 | 199.7565 | 300431- | | N/A | 139.4719 | 72.6018 | N/A | 212.0736 | 106.2 | |
| | | Control- | | | | | | | | |
| | | F5 | | | | | | | | |
| | | 300431- | PES | N/A | 133.5215 | 68.6684 | N/A | 202.1899 | 101.2 | 4.7 |
| | | PES-F5-1 | | | | | | | | |
| | | 300431- | | N/A | 134.7685 | 69.3645 | N/A | 204.1330 | 102.2 | 3.7 |
| | | PES-F5-2 | | | | | | | | |
| | | 300431- | | N/A | 134.2735 | 69.4559 | N/A | 203.7293 | 102.0 | 3.9 |
| | | PES-F5-3 | | | | | | | | |
| | | 300431- | PVDF | N/A | 133.7860 | 69.0352 | N/A | 202.8211 | 101.5 | 4.4 |
| | | PVDF-F5- | | | | | | | | |
| | | 1 | | | | | | | | |
| | | 300431- | | N/A | 131.4839 | 67.2829 | N/A | 198.7668 | 99.5 | 6.3 |
| | | PVDF-F5- | | | | | | | | |
| | | 2 | | | | | | | | |
| | | 300431- | | N/A | 135.4466 | 69.9681 | N/A | 205.4147 | 102.8 | 3.1 |
| | | PVDF-F5- | | | | | | | | |
| | | 3 | | | | | | | | |
| | | 300431- | PTFE | N/A | 132.2408 | 68.1038 | N/A | 200.3446 | 100.3 | 5.5 |
| | | PTFE-F5- | | | | | | | | |
| | | 1 | | | | | | | | |
| | | 300431- | | N/A | 133.5087 | 68.8631 | N/A | 202.3718 | 101.3 | 4.6 |
| | | PTFE-F5- | | | | | | | | |
| | | 2 | | | | | | | | |
| | | 300431- | | N/A | 131.6603 | 67.8808 | N/A | 199.5411 | 99.9 | 5.9 |
| | | PTFE-F5- | | | | | | | | |
| | | 3 | | | | | | | | |
| 5.4172 | 203.1041 | 300431- | | N/A | 147.5536 | 72.7209 | N/A | 220.2745 | 108.5 | |
| | | Control- | | | | | | | | |
| | | F6 | | | | | | | | |
| | | 300431- | PES | N/A | 147.6638 | 71.7342 | N/A | 219.3980 | 108.0 | 0.4 |
| | | PES-F6-1 | | | | | | | | |
| | | 300431- | | N/A | 145.0582 | 71.1237 | N/A | 216.1819 | 106.4 | 1.9 |
| | | PES-F6-2 | | | | | | | | |
| | | 300431- | | N/A | 142.7919 | 69.3912 | N/A | 212.1831 | 104.5 | 3.7 |
| | | PES-F6-3 | | | | | | | | |
| | | 300431- | PVDF | N/A | 142.9921 | 69.1378 | N/A | 212.1298 | 104.4 | 3.7 |
| | | PVDF-F6- | | | | | | | | |
| | | 1 | | | | | | | | |
| | | 300431- | | N/A | 143.1937 | 69.2421 | N/A | 212.4358 | 104.6 | 3.6 |
| | | PVDF-F6- | | | | | | | | |
| | | 2 | | | | | | | | |
| | | 300431- | | N/A | 142.2376 | 68.2638 | N/A | 210.5014 | 103.6 | 4.4 |
| | | PVDF-F6- | | | | | | | | |
| | | 3 | | | | | | | | |
| | | 300431- | PTFE | N/A | 144.3138 | 70.8768 | N/A | 215.1906 | 106.0 | 2.3 |
| | | PTFE-F6- | | | | | | | | |
| | | 1 | | | | | | | | |
| | | 300431- | | N/A | 144.6015 | 71.7670 | N/A | 216.3685 | 106.5 | 1.8 |
| | | PTFE-F6- | | | | | | | | |
| | | 2 | | | | | | | | |
| | | 300431- | | N/A | 142.8155 | 69.3928 | N/A | 212.2083 | 104.5 | 3.7 |
| | | PTFE-F6- | | | | | | | | |
| | | 3 | | | | | | | | |
| 9.0341 | 198.0798 | 300431- | | N/A | 134.3125 | 61.6472 | N/A | 195.9597 | 98.9 | |
| | | Control- | | | | | | | | |
| | | F7 | | | | | | | | |
| | | 300431- | PES | N/A | 136.5587 | 63.1978 | N/A | 199.7566 | 100.8 | 1.9 |
| | | PES-F7-1 | | | | | | | | |
| | | 300431- | | N/A | 135.7243 | 62.6135 | N/A | 198.3378 | 100.1 | 1.2 |
| | | PES-F7-2 | | | | | | | | |
| | | 300431- | | N/A | 135.6043 | 63.0179 | N/A | 198.6222 | 100.3 | 1.4 |
| | | PES-F7-3 | | | | | | | | |
| | | 300431- | PVDF | N/A | 136.3810 | 62.5991 | N/A | 198.9801 | 100.5 | 1.5 |
| | | PVDF-F7- | | | | | | | | |
| | | 1 | | | | | | | | |
| | | 300431- | | N/A | 136.0317 | 62.4804 | N/A | 198.5122 | 100.2 | 1.3 |
| | | PVDF-F7- | | | | | | | | |
| | | 2 | | | | | | | | |
| | | 300431- | | N/A | 153.8502 | 74.9721 | N/A | 228.8222 | 115.5 | 16.8 |
| | | PVDF-F7- | | | | | | | | |
| | | 3 | | | | | | | | |
| | | 300431- | PTFE | N/A | 136.2354 | 61.9931 | N/A | 198.2285 | 100.1 | 1.2 |
| | | PTFE-F7- | | | | | | | | |
| | | 1 | | | | | | | | |
| | | 300431- | | N/A | 135.5014 | 62.6682 | N/A | 198.1696 | 100.0 | 1.1 |
| | | PTFE-F7- | | | | | | | | |
| | | 2 | | | | | | | | |
| | | 300431- | | N/A | 137.3877 | 63.5045 | N/A | 200.8922 | 101.4 | 2.5 |
| | | PTFE-F7- | | | | | | | | |
| | | 3 | | | | | | | | |
| 9.0341 | 198.0798 | | PES, re- | | 137 | 63 | | 200 | 101.0 | 2.1 |
| | | | filtered | | 136 | 63 | | 199 | 100.5 | 1.6 |
| | | | from stock | | 136 | 63 | | 199 | 100.5 | 1.6 |

TABLE 5
Formulations with low, middle, and high concentrations of treprostinil.
WFI is water for injection. BAC is benzalkonium chloride.

| | | | |
|---|---|---|---|
| | Low | Middle | High |
| Components | (mg/mL) | (mg/mL) | (mg/mL) |

Formulation Matrix (Final)

| | | | |
|---|---|---|---|
| Trep (mg/mL) | 1.2 | 5.4 | 9.0 |
| Sodium Hydroxide (mg/mL) | 0.20 | 6.5 | 6.5 |
| Sodium Chloride (mg/mL) | 6.5 | 6.3 | 6.3 |
| Sodium Citrate (mg/mL) | 6.3 | 0.69 | 1.13 |
| 50% BAC Sol., (%) | 0.020 | 0.020 | 0.020 |
| Actual BAC in Formulation (%) | 0.010 | 0.010 | 0.010 |

Component amounts (mg)

| | | | |
|---|---|---|---|
| Trep (mg) | 240.0 | 1080.0 | 1800.0 |
| Sodium Hydroxide (mg) | 40.0 | 1300.0 | 1300.0 |
| Sodium Chloride (mg) | 1300.0 | 1260.0 | 1260.0 |
| Sodium Citrate (mg) | 1260.0 | 138.0 | 226.0 |
| BAC (mg) | 40.0 | 40.0 | 40.0 |
| WFI (mL) | 200 | 200 | 200 |
| Final pH: | 6.62 | 6.60 | 6.59 |

TABLE 6
Formulations with different concentrations of treprostinil. Benzalkonium
chloride is included as a preservative in the formulations.

| Component | Function | Treprostinil Inhalation Spray per mL (Proposed SAD Study Strengths) | | | | | | | |
|---|---|---|---|---|---|---|---|---|---|
| Treprostinil | Drug | 1.5 mg | 3 mg | 4.5 mg | 6 mg | 7.5 mg | 9 mg | 10.5 mg | 12 mg |
| | Substance | | | | | | | | |
| Sodium Hydroxide | Buffering | 0.18 | 0.37 | 0.55 | 0.74 | 0.92 | 1.1 | 1.3 | 1.5 |
| | Agent | | | | | | | | |
| Sodium Chloride | Osmotic | 6.5 mg | 6.5 mg | 6.5 mg | 6.5 mg | 6.5 mg | 6.5 mg | 6.5 mg | 6.5 mg |
| | Agent | | | | | | | | |
| Sodium Citrate | Buffering | 6.3 mg | 6.3 mg | 6.3 mg | 6.3 mg | 6.3 mg | 6.3 mg | 6.3 mg | 6.3 mg |
| Dihydrate | Agent | | | | | | | | |
| 1N Hydrochloric | Buffering | As | As | As | As | As | As | As | As |
| Acid | Agent | Required | Required | Required | Required | Required | Required | Required | Required |
| Benzalkonium | Bioburden | 0.01% | 0.01% | 0.01% | 0.01% | 0.01% | 0.01% | 0.01% | 0.01% |
| Chloride | Suppressant | w/w | w/w | w/w | w/w | w/w | w/w | w/w | w/w |
| Water for Injection | Solvent | QS to 1.0 | QS to 1.0 | QS to 1.0 | QS to 1.0 | QS to 1.0 | QS to 1.0 | QS to 1.0 | QS to 1.0 |
| | | mL | mL | mL | mL | mL | mL | mL | mL |

### Example 2

Described herein are preparation of various preserved formulation strengths using treprostinil potassium as API intended for dosing with a soft mist inhaler (SMI).

Treprostinil Potassium formulations of varying strengths were prepared with preservatives benzalkonium chloride (BKC) and metacresol (3-methylphenol). These formulations may be used for inhalation or subcutaneous delivery of treprostinil for treating cardiopulmonary diseases including Pulmonary Arterial Hypertension (PAH), Pulmonary Hypertension-Interstitial Lung Disease (PH-ILD), and/or Idiopathic Pulmonary Fibrosis (IPF). The formulations of treprostinil and treprostinil potassium are shown below:

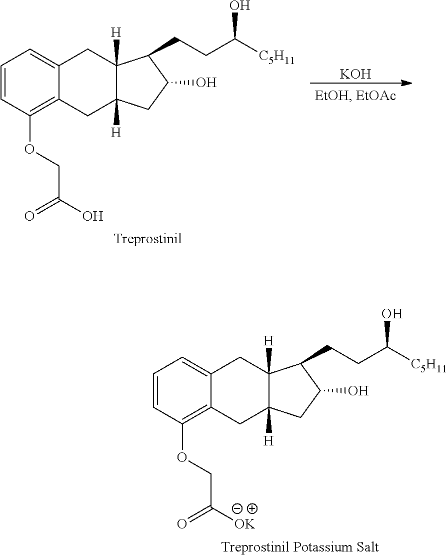

There are multiple benefits to selection of treprostinil potassium over treprostinil or other treprostinil salts. Treprostinil potassium does not require an in situ salt formation step by addition of base (NaOH or KOH). Further, treprostinil potassium is free-flowing and can be handled easily unlike treprostinil sodium salt which is hygroscopic. Further still, the amount of 1N HCl needed to adjust the final pH of the formulation was very low compared to treprostinil sodium-based formulations due to absence of excess base (like NaOH). Because of the absence of excess base in the formation of treprostinil potassium, the formation of undesired excess sodium chloride is controlled, which could provide for better control of osmolality.

### Formation of Treprostinil Potassium

Treprostinil potassium has a chemical formula of C₂₃H₃₃KO₅. Treprostinil potassium was made by the following protocol:

Potassium hydroxide (38.69 g, 608 mmol, 88.2% purity) and ethanol (400 mL) were added to a reactor. The resulting suspension was stirred at 45° C. until a clear solution resulted. Next, treprostinil (UT-15) (250 g, 640 mmol) was added in portions with constant stirring. Ethanol (100 mL) was used to rinse the UT-15 transfer container and the washings were added to the reactor. The resulting reaction mixture was stirred for 30 min. Ethyl acetate was added dropwise slowly through an addition funnel maintaining the reaction temperature between 40-45° C. The cloud point was observed when about 2000 mL of ethyl acetate was added into the reaction mixture. The cloudy suspension was stirred for 30 min and additional ethyl acetate (500 mL) was added dropwise. The stirrer was set to cool to 25° C. and the reaction mixture was stirred at 25° C. temperature for 3 h. After 3 h, the reaction mixture was filtered through a Buchner funnel using custom cut cloth filter and the solid residue obtained was washed with ethyl acetate (2×750 mL). The solid residue was air dried using a vacuum for 30 min and transferred to glass trays (wet cake 323.2 g) and dried overnight in a vacuum oven (until constant weight) to give treprostinil potassium salt (223.6 g, 85.8% yield) (100% HPLC purity, melting point: 181-183° C.). The treprostinil potassium salt was characterized by IR, 1H, 13C NMR and elemental analysis (Calc for C₂₃H₃₃KO₅: C, 64.45; H, 7.76; K; 9.12. Found: C, 63.49; H, 7.82; K, 9.35). The treprostinil potassium salt was found to have 1.63% water by Karl Fischer titration.

Treprostinil Potassium Formulations with BKC

The primary aim is to develop base formulations at 1 mg/mL (low strength), 6 mg/mL (middle strength) and 12 mg/mL (high strength) concentrations. Without wishing to be bound by theory, this range of formulations could enable the creation of intermediate strength formulations, as detailed in the discussion section. Successfully formulating treprostinil potassium at the above strengths on a 250 g scale provides a foundation for creating additional strength formulations within the bracket to allow for customization of treprostinil dosages based on device characterization testing. The osmolality of the prepared formulations was determined.

TABLE 7
Formulations of treprostinil potassium.

| Material | Formulation 1 | | Formulation 2 | | Formulation 3 | |
|---|---|---|---|---|---|---|
| Treprostinil | 1 | mg/mL | 6 | mg/mL | 12 | mg/mL |
| Potassium | | | | | | |
| Sodium | 6.5 | mg/mL | 6.5 | mg/mL | 6.5 | mg/mL |
| Chloride | | | | | | |
| Sodium Citrate | 6.3 | mg/mL | 6.3 | mg/mL | 6.3 | mg/mL |
| Dihydrate | | | | | | |
| 50% BKC | 0.02% | w/w | 0.02% | w/w | 0.02% | w/w |
| (Effective) | (0.01% | w/w) | (0.01% | w/w) | (0.01% | w/w) |
| 1N HCl | As Required | | As Required | | As Required | |
| Final pH | ~6.6 | | ~6.6 | | ~6.6 | |
| Water for | QS to 250 g | | QS to 250 g | | QS to 250 g | |
| Injection (WFI) | | | | | | |

While each formulation varies, the methods for preparing the formulations follows the same steps, adjusting where necessary for the correct parameter. The method can be sized up accordingly for a larger batch of inhalation formulation solution.

The following is the formulation procedure for a Formulation 1 (1 mg/mL):
1. Weigh 225 g of WFI into a 500 mL polypropylene beaker equipped with a stir bar and a calibrated pH probe.
2. Weigh 250 mg of treprostinil potassium and transfer to the beaker. Stir for ˜5 minutes.
3. Weigh 1625 mg of sodium chloride and transfer to the beaker. Stir for ˜5 minutes.
4. Weigh 1575 mg of sodium citrate dihydrate and transfer to the beaker. Stir for ˜5 minutes.
5. Weigh 50 mg of 50% BKC in a 4 mL vial and transferred to the beaker using 2×˜2 mL WFI. Stir for ˜2 minutes.
6. Add 1N HCl (425 μL) in 25 to 100 μL increments to obtain a final pH of 6.630.
7. The above prepared formulation was QS (quantum satis) to 250 g final weight using WFI. The pH of this clear solution was found to be 6.645.
8. Filter the formulation into a media bottle through 0.2 μm polyethersulfone (PES) membrane filter.
9. Formulation should remain clear overnight at room temperature.

### Results

Osmolality of the prepared formulations was determined using an Osmometer. The values are given in Table 8.

TABLE 8
Osmolality of the Prepared Formulations

| Formulation. | | |
|---|---|---|
| No. | Strength | Osmolality |

| | | | |
|---|---|---|---|
| 1. | 1 | mg/mL | 276 mOsm/kg |
| 2. | 6 | mg/mL | 292 mOsm/kg |
| 3. | 12 | mg/mL | 301 mOsm/kg |

Treprostinil Potassium Formulations with Metacresol

Treprostinil potassium (110 mg) was dissolved in 95 mL of water and stirred until clear solution. To this sodium citrate (desired amount) was added and stirred for 10 mins. Then sodium chloride (530 mg) was added and stirred for 10 min. To this Metacresol (300 mg) was added and stirred for 10 mins. The pH of the resulting solution was adjusted to pH˜6.3 using desired amount of 1 N HCl. The final formulation was QS to 100 mL using water to obtain a clear formulation.

TABLE 9
Treprostinil potassium formulations with Metacresol

| Material | Formulation 1 | | Formulation 2 | |
|---|---|---|---|---|
| Treprostinil Potassium | 1.1 | mg/mL | 1.1 | mg/mL |
| Sodium Chloride | 5.3 | mg/mL | 5.3 | mg/mL |
| Sodium Citrate | 6.3 | mg/mL | 3.15 | mg/mL |
| Dihydrate | | | | |
| Metacresol | 3 | mg/mL | 3 | mg/mL |

| | | |
|---|---|---|
| 1N HCI | As Required | As Required |
| Final pH | ~6.3 | ~6.3 |
| Water | QS to 100 mL | QS to 100 mL |

### Example 3

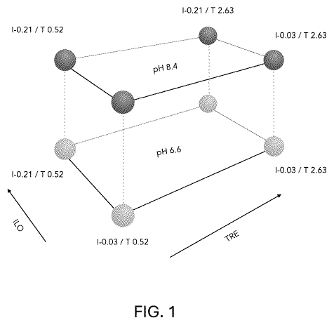

Described herein are preparation of various formulation strengths using treprostinil and iloprost. FIG. 1 shows a 3-D representation of different combination formulations (“I” represents “iloprost” and “T” represents “treprostinil”) at 2 levels of pH, 6.6 or 8.4, where the concentration of iloprost varies from 0.03 mg/ml to 0.21 mg/ml and the concentration of treprostinil varies from 0.52 mg/ml to 2.63 mg/ml. Table 10 lists specific formulations 1-9 comprising selected concentrations of iloprost and treprostinil at a pH of 6.6 or 8.4.

The combination of treprostinil and iloprost—both prostacyclin analogs—could have a synergistic effect on IP receptors potentially enhancing vasodilation and reducing pulmonary arterial pressure more effectively than either compound alone. Treprostinil and iloprost both act as agonists of the IP receptor, which, when activated, leads to the production of cyclic AMP (CAMP). This second messenger mediates vasodilation by relaxing vascular smooth muscle cells. Iloprost has shorter half-life and faster onset of action, while treprostinil has longer half-life and slower onset of action. A combination of iloprost and treprostinil could potentially provide a faster onset and a sustained physiological response.

In some aspects, formulations comprising treprostinil and iloprost comprise BKC at a concentration of 0.005% (w/w) to 0.2% (w/w).

TABLE 10
Iloprost-Treprostinil Formulation

| | Iloprost | Treprostinil | |
|---|---|---|---|
| Formulation # | (mg/mL) | (mg/mL) | pH |
| 1 | 0.30 | 0.32 | 8.4 |
| 2 | 0.30 | 3.78 | 8.4 |
| 3 | 0.03 | 0.32 | 8.4 |
| 4 | 0.03 | 3.78 | 8.4 |
| 5 | 0.30 | 0.32 | 6.6 |
| 6 | 0.30 | 3.78 | 6.6 |
| 7 | 0.03 | 0.32 | 6.6 |
| 8 | 0.03 | 3.78 | 6.6 |
| 9 | 0.14 | 1.73 | 7.5 |

### Example 4

Described herein is a study protocol to evaluate the pharmacokinetics, safety, and tolerability of treprostinil soft mist inhaler spray (“Treprostinil SMI”) in healthy participants.

This study aims to evaluate treprostinil inhalation spray delivered as treprostinil SMI. Treprostinil SMI provides patients with an inhalation device that is small and portable. Treprostinil SMI delivers treprostinil inhalation spray to the lung and is available in multiple strengths.

The study indications are evaluated are pulmonary arterial hypertension and pulmonary hypertension associated with interstitial lung disease.

### Study Design

A Phase 1, single ascending dose study enrolls up to 48 healthy adult participants. The study design consists of a Screening Phase followed by a Treatment Phase with up to 8 cohorts, and each cohort enrolls 6 participants. During the Screening Phase, participants who meet the study criteria are eligible for enrollment. Each participant receives a single administering event of treprostinil SMI comprising a single breath if tolerated, and serial blood samples are taken for PK analysis. Each cohort receives a subsequently higher dose than the previous cohort until protocol-defined stopping criteria are met. The study is an open-label study. Participants are sequentially enrolled into up to 8 cohorts of 6 participants each.

### Drug Formulation

Treprostinil are delivered by a soft mist inhaler in a combination drug-device product that consists of the inhaler and an associated cartridge with a propellant-free, liquid-based formulation of treprostinil inhalation spray. The combination drug-device product is referred to as Treprostinil SMI Inhalation Spray (“treprostinil SMI”).

The device is a handheld, pocket-sized, oral inhalation device that uses mechanical energy to generate a slow-moving aerosol of medication from a metered dose of the study drug formulation. A single-use study drug cartridge is manually inserted into the soft mist inhaler and primed prior to dosing. The device is then actuated, and a soft mist is discharged from the inhaler for the participant to inhale.

Treprostinil SMI is supplied with single-use cartridges, each containing between 18 to 144 micrograms (mcg) of treprostinil (Table 11). Each cohort receives a subsequently higher dose than the previous cohort until protocol-defined stopping criteria are met. The doses were selected based on anticipated similarity in exposure to other inhaled treprostinil formulations.

Treprostinil SMI is expected to provide an additional delivery method of inhaled treprostinil, which may be more convenient for some patients with PAH and PH-ILD. Treprostinil SMI is a new formulation composed of treprostinil in a sodium citrate buffer solution with benzalkonium chloride as an excipient.

Benzalkonium chloride is an excipient used in other approved inhalation products and is listed in the FDA Inactive Ingredient Database (CAS Number 8001545; UNII F5UM2KM3W7) at concentrations for inhalation products up to 0.02% w/w.

TABLE 11
Treprostinil Dose in Study

| | Treprostinil SMI | Cartridge Concentration |
|---|---|---|
| Cohort | (mcg) | (mg/mL) |
| 1 | 18 | 1.5 |
| 2 | 36 | 3 |
| 3 | 54 | 4.5 |
| 4 | 72 | 6 |
| 5 | 90 | 7.5 |
| 6 | 108 | 9 |
| 7 | 126 | 10.5 |
| 8 | 144 | 12 |

### Study Objectives and Endpoints

Primary Endpoint: To evaluate the systemic exposure and pharmacokinetics of treprostinil administered as treprostinil SMI.

Secondary Endpoint: To evaluate the safety and tolerability of treprostinil SMI. This is evaluated by determining the incidence and severity of reported adverse events, as well as changes from baseline in vital signs, clinical laboratory tests, electrocardiograms, and physical examinations.

### Pharmacokinetic Assessments

In all cohorts, serial blood samples are taken via an in-dwelling intravenous catheter or by direct venipuncture on Day 1 at pre-dose and at 5, 10, 15, 30, 45, 60, 90, 120, 180, 240, and 300 minutes after study drug administration. Pre-dose samples are collected before administration of study drug. Blood samples are analyzed for treprostinil plasma concentrations using validated bioanalytical LC-MS methods. PK parameters are obtained from the resulting plasma drug concentration-time data.

### Safety and Tolerability Assessments

Vital signs are assessed for each cohort at all study visits, including the end of study or early termination visit. On Day 1, vital signs are assessed at pre-dose (within 1 hour prior to planned dosing time) and 15 minutes and 1 and 4 hours post-dose (±10 minutes).

Vital signs measured includes height (Screening only), weight, blood pressure (systolic and diastolic), peripheral (radial or brachial artery) heart rate, respiration rate, and temperature.

Clinical laboratory panels (Table 12) are assessed for all participants during the Screening Phase, at Baseline (prior to treatment), and at the end of study or early termination visit.

TABLE 12
Clinical Laboratory Panels.

| Chemistry Panel | Hematology Panel |
|---|---|
| Albumin | Absolute neutrophil count |
| Alkaline phosphatase | Erythrocytes |
| Anion gap | Hemoglobin |
| Total bilirubin | Hematocrit |
| Calcium | Leukocytes |
| Carbon dioxide | Mean corpuscular hemoglobin |
| Chloride | Mean corpuscular hemoglobin |
| Total cholesterol | concentration |
| Creatinine | Mean corpuscular volume |
| Gamma glutamyltransferase | Platelet count |
| Globulin | Red cell distribution width |
| Glucose | White blood cell count |
| Lactate dehydrogenase | White blood cell count differential |
| Phosphorus | |
| Potassium | |
| Total protein | |
| Alanine aminotransferase | |
| Aspartate aminotransferase | |
| Sodium | |
| Triglycerides | |
| Uric acid | |
| Creatinine | |
| Calcium | |
| Albumin | |
| Glucose | |
| Sodium | |
| Potassium | |
| Carbon dioxide | |
| Chloride | |

A 12-lead ECG are obtained from all participants during the Screening Phase, at Baseline, 30 minutes post-dose on Day 1, and at the end of study or early termination visit.

### Adverse Events

All adverse events (AE) are documented from the time of informed consent until the participant is either discontinued from the study or all end of study or early termination assessments have been completed.

An AE is any untoward medical occurrence in a patient or clinical investigation participant administered a pharmaceutical product that does not necessarily have to have a causal relationship with this treatment. An AE may include an intercurrent illness, injury, or any other concomitant impairment of the participant's health, as well as abnormal laboratory findings if deemed to have clinical significance. AEs may also include worsening of an existing symptom or condition or post-treatment events that occur as a result of protocol-mandated procedures.

A serious adverse event is an AE occurring during the clinical study that results in any of the following outcomes: death, life-threatening AE, inpatient hospitalization or prolongation of existing hospitalization, a persistent or significant disability/incapacity, or a congenital anomaly/birth defect.

Adverse events will be followed.

### Results and Statistics

Two study populations are defined: a Safety Population and a PK Population. The Safety Population will include all participants who receive any amount of study drug. The PK Population will include all participants who receive study drug and who have sufficient study drug concentration-time data to derive noncompartmental PK parameters.

Descriptive statistics are used to summarize the baseline characteristics, safety and tolerability endpoints, and PK parameters.

The data is presented by dose cohort.

Plasma samples are analyzed for treprostinil concentrations using validated bioanalytical methods. The concentration-time plots for treprostinil are presented by dose cohort. Individual and mean treprostinil plasma concentration data are summarized by dose cohort. A noncompartmental model is used to calculate the PK parameters for treprostinil inhalation spray. The PK parameters include maximum plasma concentration, time of maximum plasma concentration, half-life, elimination rate constant, apparent plasma clearance, apparent volume of distribution, and AUC_{0-inf} (AUC from zero to time infinity). PK parameters are listed and summarized by dose cohort. The dose proportionality is explored.

Statistical summaries of safety data are descriptive and exploratory in nature, focusing on the incidence of AEs. Treatment-emergent AEs is summarized by treprostinil SMI dose cohort. Other safety variables, including clinical laboratory parameters, ECG parameters, and vital signs, are summarized for the original value and the change from baseline values.

### Example 5—Pharmacokinetic Data

Across all 8 study cohorts, a total of 49 participants entered the study and contributed to the PK data analysis. Serial blood samples were taken via an in-dwelling intravenous catheter or by direct venipuncture by qualified study personnel on Day 1 at pre-dose and at 5, 10, 15, 30, 45, 60, 90, 120,180,240, and 300 minutes after study drug administration. Blood samples were analyzed for treprostinil plasma concentrations using validated bioanalytical LC-MS methods. PK parameters were obtained from the resulting plasma drug concentration-time data.

PK data were analyzed. Plasma concentrations of treprostinil SMI were used to calculate the area under the curve from time Oto 5 hours (AUCo-s), area under the curve from time Oto infinity (AUCo-inf), maximum observed plasma concentration (Cnax), time of maximum plasma concentration (Tmax), and half-life (t112) for each dose cohort of treprostinil SMI. AUC was approximately dose-proportional.

### Treprostinil SMI PK Data Versus Historical Tyvaso Nebulizer Comparator

The PK data of nebulized Tyvaso from the TIP-PH-102 study was used as a historical comparator. In Study TIP-PH-102, nebulized Tyvaso was the reference treatment and up to 36 healthy participants received a single dose of nebulized Tyvaso at doses of 18 mcg (3 breaths), 54 mcg (9 breaths), and 72 mcg (12 breaths) of treprostinil (Table 13). PK samples were collected at pre-dose and at 5, 10, 15, 30, 45, 60, 90, 120,180,240, and 300 minutes after administration of nebulized Tyvaso.

TABLE 13
Nebulized Tyvaso Doses in TIP-PH-102

| Nebulized Tyvaso Dose (mcg) | No. of Participants |
|---|---|
| 18 (3 nebulizer breaths) | 34 |
| 54 (6 nebulizer breaths) | 34 |
| 72 (12 nebulizer breaths) | 35 |

### AUC0.5 and Cmax

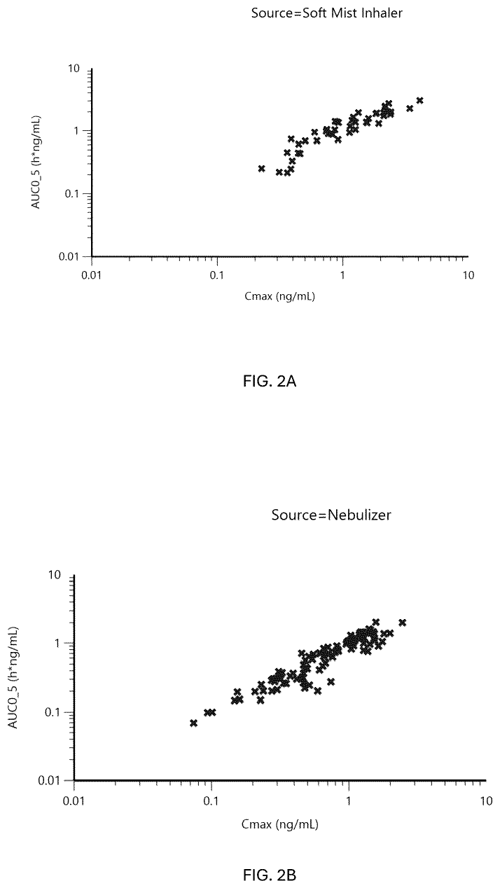

The log-log plots of the relationship between AUCo-s as the dependent variable and Cmax as the independent variable for the treprostinil SMI formulation (SMI-PH-101) and the historical Tyvaso nebulizer comparator (TIP-PH-I 02) across all dosing levels are shown in FIGS. 2A and 2B. A linear relationship between the 2 variables was observed with a slope of about 1 due to the short half-life of treprostinil.

### CLIF

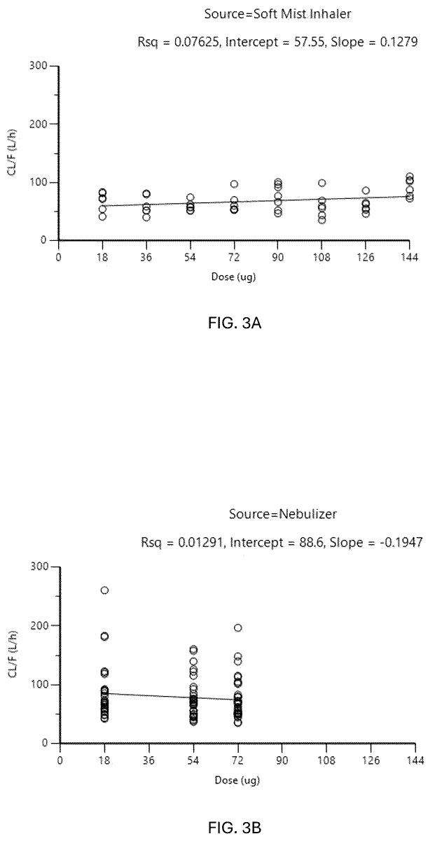

D Plots of the apparent clearance (clearance [CL] divided by the bioavailability [F]) are shown in FIGS. 3A and 3B. CL/F values for treprostinil SMI were largely 100 L/h and narrowly distributed due to low intersubject variability for this device. In contrast, several CL/F values for the Tyvaso nebulizer were greater than 100 L/h at each dose level reflecting a greater degree of intersubject variability.

Table 14 (below) summarizes the descriptive statistics comparing the CL/F values for soft mist inhaler and the Tyvaso nebulizer including all participants at all dose levels for each device at each dose level. These data are compiled from 49 healthy participants receiving treprostinil SMI over the dose range of 18 mcg (1.5 mg/mL) to 144 mcg (12.0 mg/mL) (SMI-PH-101) and 98 healthy participants receiving Tyvaso nebulizer doses ranging from 18 to 72 mcg. The overall CV % for treprostinil SMI is considerably lower at 28.1% compared to the value of 48.9% for the Tyvaso nebulizer. The lower variability demonstrated by the SMI formulation for CL/F would likely support greater precision in clinical patient dosing with the SMI.

TABLE 14
Descriptive Statistics for CL/F for Treprostinil
SMI and the Tyvaso Nebulizer - All Participants

| | | CL/F |
|---|---|---|
| Source | Stats | (mcg/(h*ng/mL)) |
| SMI | N | 49 |
| | Mean | 67.939 |
| | CV % | 28.1 |
| | Min | 35.50 |
| | Median | 62.69 |
| | Max | 110.62 |
| | Geometric Mean | 65.392 |
| Tyvaso Nebulizer | N | 98 |
| | Mean | 79.155 |
| | CV % | 48.9 |
| | Min | 35.51 |
| | Median | 68.81 |
| | Max | 260.24 |
| | Geometric Mean | 72.259 |

### Relative Bioavailability for Treprostinil SM/Versus Tyvaso Nebulizer

A population estimate of the relative bioavailability (Frel) was obtained by using the comparison of the CL/F values as presented in the equation below. Since CL/Fis calculated from the dose administered divided by AUC, the ratio of the CL/F values provides an estimate of the relative bioavailability for each formulation.

Frei=(CLIFNEBULIZER)/(CL/FsMI)

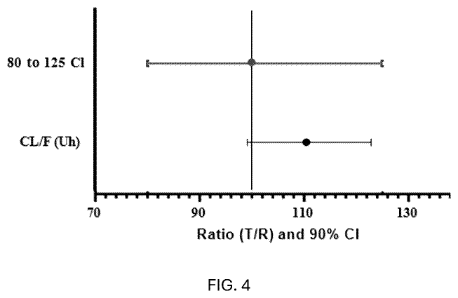

FIG. 4 and Table 15 (below) show the results of the comparative bioavailability analysis using CL/F as the dependent variable and indicate that the soft mist inhaler had 10.5% greater bioavailability and that the geometric least squares mean ratio (GLSMR) for CL/F (Nebulizer vs. SMI) has a 90% confidence interval of 99.24 to 123.04.

TABLE 15
Comparative Bioavailability using CL/F for All Participants
at All Doses for Tyvaso Nebulizer vs. Treprostinil SMI

| | | | | | | | | |
|---|---|---|---|---|---|---|---|---|
| | | | | Reference | Test | Ratio % | 90% CI | 0% CI |
| Dependent | Units | ‘Reference’ | ‘Test’ | GLSM | GLSM | Reference | Lower | Upper |

| | | | | | | | |
|---|---|---|---|---|---|---|---|
| Ln(CL/F) | mcg/(h*ng/mL) | Tyvaso | 65.4 | 72.3 | 110.50 | 99.24 | 123.041 |
| | | Nebulizer | | | | | |

### Treprostinil SMI Dose Modeling

Dose modeling was performed utilizing treprostinil SMI PK data from the SMI-PH-101 study and nebulized Tyvaso PK data from the historical comparator, the TIP-PH-102 study. Based on the 10.5% greater bioavailability from the CL/F calculations of the SMI formulation compared to the Tyvaso nebulizer formulation, the proposed doses for the SMI-PH-102 study were determined by multiplying the SMI-PH-101 doses by 89.5% (1-0.105) and are listed in Table 16 (below). The resultant treprostinil SMI cartridge strengths of 1.34 mg/mL, 4.02 mg/mL, and 6.70 mg/mL will be administered in the SMI-PH-102 study to deliver doses expected to be comparable to 18 mcg (3 breaths), 54 mcg (9 breaths), and 90 mcg (15 breaths) of nebulized Tyvaso.

TABLE 16
Proposed SMI-PH-102 Treprostinil SMI Cartridge
Concentrations from Dose Modeling

| SMI-PH-101 | | SMI-PH-102 | Comparable | Equivalent |
|---|---|---|---|---|
| Cartridge | | Cartridge | Tyvaso | Breaths of |
| Concentration | | Concentration | Nebulizer Dose | Nebulized |
| (mg/mL) | Frei(%) | (mg/mL) | (mcg) | Tyvaso |
| 1.5 | 110.5 | 1.34 | 18 | 3 breaths |
| 4.5 | | 4.02 | 54 | 9 breaths |
| 7.5 | | 6.70 | 90 | 15 breaths |

### Pivotal Relative Bioavailability PK Study-SMI-PH-102

Applicant to move forward with the pivotal relative bioavailability PK study, SMI-PH-102, to compare treprostinil exposure between the planned commercial strengths of treprostinil SMI and nebulized Tyvaso. Based on the above dose modeling data, treprostinil SMI cartridge strengths of 1.34 mg/mL, 4.02 mg/mL and 6.70 mg/mL, intended to deliver doses comparable to 18 mcg (3 breaths), 54 mcg (9 breaths), and 90 mcg (15 breaths) of nebulized Tyvaso, will be used in the SMI-PH-102 study (Table 17, below).

TABLE 17
SMI-PH-102 Dosing of Nebulized Tyvaso and Treprostinil SMI

| Comparable Doses ofTyvaso | SMI PH-102 Cartridge |
|---|---|
| Nebulizer (mcg) | Concentration (mg/mL) |
| 18 | 1.34 |
| 54 | 4.02 |
| 90 | 6.70 |

The total sample size is planned to be 36 participants (6 participants per sequence in 6 sequences). The PK data obtained from a previous study using Tyvaso (TIP-PH-102) indicate that the coefficient of variation was 22% for AUCinf, 25% for AUClast, and 27% for Cₘₐₓ-Assuming a coefficient of variation of 28% and that the true ratio is 1, 35 participants are needed to target 90% Cls for geometric mean ratios of treprostinil SMI versus Tyvaso for AUCnr, AUClast, and Cmax within 80% to 125% with at least 90% power.

The SMI-PH-102 protocol and supportive manufacturing information for the planned cartridge concentrations will be submitted to the IND following incorporation of any Agency feedback in the meeting written responses.

### Example 6—Stability Data of Treprostinil Formulations

Stability data from clinical GMP lots of treprostinil inhalation spray manufactured at United Therapeutics Corporation (Silver Spring, MD) demonstrated stability of treprostinil inhalation spray formulations past 3 months for varying treprostinil and BKC concentrations. Specifically, all batches tested following 3 month storage under acceptable conditions (25° C./60% RH) demonstrated impurities below 0.02% total impurities, which is well below the 3% acceptance criteria. Moreover, the formulations retained pH and bioburden values well within the acceptance criteria values. Finally, HPLC data demonstrated that all treprostinil formulations retained acceptable levels of Benzalkonium Chloride. Accordingly, the treprostinil formulations described herein demonstrate suitable stability and purity values sufficient for extended storage without the production of impurities or variances in treprostinil concentration and pH.

TABLE 18
Treprostinil Inhalation Spray Clinical GMP Stability Studies

| | Strength | | Benzalkonium | 25° C./60% RH | |
|---|---|---|---|---|---|
| | (Treprostinil | | chloride | Timepoint | Purpose |
| Batch # | Concentration) | | concentration | (Long-Term) | of Study |
| 1501253 | 1.5 | mg/mL | 0.1 mg/ml (0.01%) | 3 Months | Clinical GMP |
| 1501258 | 3.0 | mg/mL | 0.1 mg/ml (0.01%) | 3 Months | Clinical GMP |
| 1501260 | 4.5 | mg/mL | 0.1 mg/ml (0.01%) | 3 Months | Clinical GMP |
| 1501261 | 6.0 | mg/mL | 0.1 mg/ml (0.01%) | 3 Months | Clinical GMP |
| 1501262 | 7.5 | mg/mL | 0.1 mg/ml (0.01%) | 3 Months | Clinical GMP |
| 1501263 | 9.0 | mg/mL | 0.1 mg/ml (0.01%) | 3 Months | Clinical GMP |
| 1501264 | 10.5 | mg/mL | 0.1 mg/ml (0.01%) | 3 Months | Clinical GMP |
| 1501265 | 12.0 | mg/mL | 0.1 mg/ml (0.01%) | 3 Months | Clinical GMP |

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. An inhalation formulation comprising a preservative and treprostinil or a pharmaceutically acceptable salt thereof and a pharmaceutically acceptable carrier, wherein the treprostinil is at a concentration of about 100 mg/mL to about 25 mg/mL, and wherein the formulation is in a solution form.

2. The inhalation formulation of claim 1, further comprising a benzalkonium chloride (BKC) preservative and a pharmaceutically acceptable carrier, and wherein the formulation is in a solution form.

3. The inhalation formulation of claim 1, wherein the treprostinil is at a concentration of about 1.2 mg/mL to about 12 mg/mL.

4. The inhalation formulation of claim 1, further comprising a second prostacyclin.

5. The inhalation formulation of claim 4, wherein the second prostacyclin is iloprost or a pharmaceutically acceptable salt thereof.

6. The formulation of claim 5, wherein the concentration of iloprost ranges from about 100 μg/mL to about 500 g/mL.

7. The inhalation formulation of claim 2, wherein the BKC is at a concentration of about 0.005% (w/w) to about 0.2% (w/w).

8. The inhalation formulation of claim 1, wherein the pH of the solution is about 5.5 to about 8.5.

9. The inhalation formulation of claim 1, wherein the viscosity of the solution is about 0.8 cP to about 1.2 cP.

10. The inhalation formulation of claim 1, further comprising sodium hydroxide, sodium chloride, sodium citrate dihydrate or sodium citrate, and water.

11. The inhalation formulation of claim 1, wherein the treprostinil salt is treprostinil sodium.

12. The inhalation formulation of claim 1, wherein the treprostinil salt is treprostinil potassium.

13. The inhalation formulation of claim 1, further comprising metacresol at a concentration of about 1 mg/mL to about 10 mg/mL.

14. A soft mist inhaler comprising an effective amount of the formulation of claim 1.

15. An inhaler comprising an effective amount of the inhalation formulation of claim 1.

16. An inhalation formulation comprising iloprost or a pharmaceutically acceptable salt thereof, treprostinil or a pharmaceutically acceptable salt thereof, and benzalkonium chloride, wherein the concentration of iloprost ranges from about 10 μg/mL to about 500 μg/mL, and wherein the concentration of treprostinil ranges from about 200 μg/mL to about 4000 μg/mL.

17. A method of treating a cardiopulmonary disorder comprising administering by inhalation to a patient in need thereof a therapeutically effective single event dose of an inhalation formulation comprising a preservative and treprostinil or a pharmaceutically acceptable salt thereof and a pharmaceutically acceptable carrier, wherein the treprostinil is at a concentration of about 100 mg/mL to about 25 mg/mL, and wherein the formulation is in a solution form.

18. The method of claim 17, wherein the administering is performed with a soft mist inhalation device.

19. The method of claim 17, wherein the cardiopulmonary disorder is selected from pulmonary hypertension and pulmonary fibrosis.

20. The method of claim 17, wherein the administering is done with an inhaler or nebulizer.
